(19) United States
(12) Patent Application Publication
(10) Pub. No.: US 2010/0043785 A1
(43) Pub. Date: Feb. 25, 2010

(54) INHALER WITH A FORWARD METERING VALVE

(75) Inventors: Joergen Rasmussen, Struer (DK); Soeren Christrup, Struer (DK)

Correspondence Address: ROBERTS MLOTKOWSKI SAFRAN & COLE, P.C.;Intellectual Property Department, P.O. Box 10064, MCLEAN, VA, 22102-8064, US

(73) Assignee: BANG & OLUFSEN MEDICOM A/S, Struer (DK)

(21) Appl. No.: 12/514,741

(22) PCT Filed: Nov. 14, 2007
(86) PCT No.: PCT/DK07/00500

§ 371 Date: May 13, 2009

(30) Foreign Application Priority Data
Nov. 14, 2006 (DK) PA 2006 1483

(51) Int. Cl. A61M 11/00; B65B 1/04

(52) U.S. Cl. 128/200.23; 141/3

(57) ABSTRACT

A metered dose inhaler for administration a liquid phase medium using a rotating metering element that transports the metered dose from the pressurized canister to the mouthpiece. The metering is improved in that there is an efficient protection against penetration of outside air, and in that the counting of the doses is improved by preventing backwards rotation of the rotating metering element.

### BACKGROUND OF THE INVENTION

1. Field of Invention

The invention is directed to generic types of inhalers.

2. Description of Related Art

In the treatment of asthma, COPD, diabetes, systemic pain etc., which can be treated by inhalation of a drug medium, inhalation devices with a bulk of medical drugs are widely used.

Normally, two different medical drug formulations are used—one providing the drug in dry powder form (dry powder inhaler=DPI), and one, where the drug is mixed into a suitable propellant in pressurized, liquid form (pressurized metered dose inhaler=pMDI). Liquid phase pressure free drugs, packaged in multi-dose blisters or small bags are also seen. The associated devices incorporate a spring loaded piston to establish a pressure within the blisters or bags.

### SUMMARY OF THE INVENTION

New regulatory issues require that both DPI's and pMDI's are equipped with a reliable dose indicator, indicating the number of doses left to the patient in the inhaler.

New developments of pharmaceutical formulations, where medical drugs are targeted to be administered via the lungs, especially in diabetes and pain relief, raise new requirements to the accuracy of dose metering and dynamic dose titration such as rapid multiple sequential release of doses within the same inhalation period.

The metering valve should preferably involve few parts and be well suited for automatic assembly and low manufacturing costs.

The pMDI

In the pMDI the medical drug is mixed into a propellant liquid and contained under pressure in a canister. To meter and release the drug in uniform doses, the canister is mounted with a metering valve i. e. as disclosed in U.S. Pat. No. 3,756,465 to Meshberg.

The common valve is a compress-and-release type of valve. From this, the more popular name “press-and-breathe” has been given to the pMDI (FIG. 1).

While the patient inhales through the mouthpiece of the pMDI (11) he/she is supposed to manually compress and release the pMDI canister (12) to obtain the inhaled drug, illustrated by the curve in FIG. 2. Initial position (21) is to the left on the curve, where the canister is in a fully extended state.

While inhaling through the mouthpiece, the canister is manually compressed (22), passing the point of release of the previously metered dose (23) until it reaches its fully compressed state (24). After a certain delay (25), the canister is released (26), passing the point of metering of the next dose (27) until it again reaches the fully extended state (28) (reset).

A number of problems are known in present pMDI devices that may result in improper dose release:
A. During inhalation the patient must be capable of overcoming the canister compression force of 30-50 N to release the previously metered dose of drug (23) (canister pressure typically 0.3-0.6 MPa). The majority of this force is required to compress a built-in return spring, strong enough to ensure the reset of the valve (28).
B. The patient must hold the pMDI upright at metering of the next dose (27)—after inhalation—otherwise he will not obtain a full dose of drug at next use. The rationale for this is difficult to understand for the patient.
C. The delay (25) must not exceed 5-10 seconds, otherwise the metering of the next dose (27) will be inaccurate and the patient will not obtain a precise dose of drug at next use.

The main reasons for inaccurate dose releases are:
1. Metering from a non-uniformly dissolved solution. The user is instructed to shake the inhaler prior to inhalation in order to dissolve the drug uniformly in the liquid solution inside the canister. If too long time elapses from shaking to metering of the next dose, the solution becomes non-uniformly dissolved, resulting in a too low or too high dose of drug metered for next use.
2. Atmospheric air trapped inside the metering chamber. By delaying the metering of next dose after delivery of the present dose, air will migrate from the outside into the metering valve. The air will be trapped inside the metering valve taking up volume, resulting in a too small metered dose.

Furthermore, there is a risk of drug leaking from the canister to the outside during the delay, as a smaller load is applied to the gaskets inside the metering valve during canister compression. This can lead to a serious lack of drug, when needed by the user.

To overcome the problem of (A) and to improve the coordination between inhalation and dose delivery, breath actuated inhalers (BAI's) have been developed. But as reset must be performed manually by the patient after inhalation, e.g., by closing the cap of the BAI, the risks of (B) and (C) are getting seriously worse.
D. If the time between doses is too long (e.g., some days) there is a risk of degradation of the metered dose (loss of prime). It is required that the patient performs priming shots, both before first use and before use if the pMDI has not been used for some days or longer.
E. Adding a reliable dose indicator to a pMDI or BAI introduces new problems. As the magnitude of the position tolerance of the canister is comparable to the canister stroke during compression and release, it is not obvious how to design a tamper-proof dose indicator which never counts too many doses AND never counts too few doses.

Canister filling issues:
F. Generally, canister mounted metering valve designs must comply with the industry practice of factory filling of the canister with drug and propellant. It is common procedure that the metering valve is mounted on the canister before filling, therefore the valve design must allow for reverse flow, when high pressure is applied to the metering valve outlet to fill the canister.

Rotational metering valves are well known from prior art relating to dry powder inhalers, such as UK Patent Application GB 2165159 to Auvinen. However, these valves are pressure and sealing free, and depend on gravity only.

Rotational dose metering devices for fluids are known from, e.g., gasoline pumps, and within the medical field some examples has been disclosed in U.S. Pat. No. 6,179,583 to Weston and U.S. Pat. No. 6,516,796 to Cox. These valves are designed to work with propellant-free liquids at low pressures, they are complicated and expensive to manufacture and have not been demonstrated to work at the typical canister pressure of 0.3-0.6 MPa.

### SUMMARY OF THE INVENTION

This invention relates to an inhaler with a sealed rotational metering valve with fixed metering cavities to be used with pressurized canister based aerosol inhalers. The invention solves several of the above mentioned problems inherent with existing pressurized aerosol inhalers:

Activation force is minimized as the metering valve does not need a preloaded return spring.

The metering valve is filled and the metered dose is released in one actuation movement after the user has placed the inhaler in upright position for oral or nasal application. Therefore a full dose will be reliably and accurately metered and problems with long term migration of a former metered dose are avoided.

The need for reset time is obviated, as the liquid in the canister will flow freely into the metering cavity.

In the unidirectional rotational motion mode, the metering valve is unambiguously well suited for a simple counter mechanism; a reliable visual dose indicator is easily attached to the valve.

An embodiment of an elastic sealing member for a ball shaped valve rotor is disclosed that allows for standard canister filling procedures. A filling procedure for a cylindrical shaped valve member is also disclosed.

The forward metering valve can be part of the drug canister/container or it can be an add-on device to the drug canister/container.

A further aspect of the invention is that the forward metering valve is extremely suitable for multi-dose operation, because the mechanical movement of the valve can be rotational, continuous and unidirectional.

### BRIEF DESCRIPTION OF THE DRAWINGS

FIG. 1 shows a conventional pMDI inhaler.

FIG. 2 shows the timing associated with a conventional inhaler.

FIG. 3a shows one embodiment of a ball shaped forward metering valve.

FIG. 3b shows one embodiment of a conically shaped forward metering valve.

FIG. 3c shows one embodiment of a cylindrically forward metering valve.

FIG. 4 shows the functional steps of the forward metering valve involved during one inhalation action.

FIG. 5 shows the positioning of an optional one-way valve.

FIG. 6 shows one the embodiments of the attachment of a dose counter wheel.

FIG. 7 shows one embodiment of a backward locking mechanism.

FIG. 8 shows one embodiment of a step locking mechanism.

FIG. 9 shows one embodiment of finger wheel actuation mechanism.

FIG. 10 shows one embodiment of a pushbutton/ratchet mechanism.

FIG. 11 shows an embodiment of a breath actuated dose release mechanism.

FIG. 12a shows an embodiment of the forward metering valve integral within a canister

FIG. 12b shows a principle of filling the canister through the forward metering valve

FIG. 12c shows a filling situation for a cylindrically shaped valve.

FIG. 12d shows the valve in 12c in its closed position after filling.

FIG. 13 shows an inhaler configuration, where a forward metering valve is driven by a battery powered motor under control of a breath activated mechanism and a control unit.

FIG. 14 shows the timing of a single dose release with a breath activated release mechanism.

FIG. 15 shows the timing of a multi dose release with a breath activated release mechanism.

FIG. 16 shows the timing of an adaptive multi dose release controlled by continuous measurement of inhalation flow.

FIG. 17 shows an inhaler structure with a rotational cylindrically shaped valve in the simplest configuration.

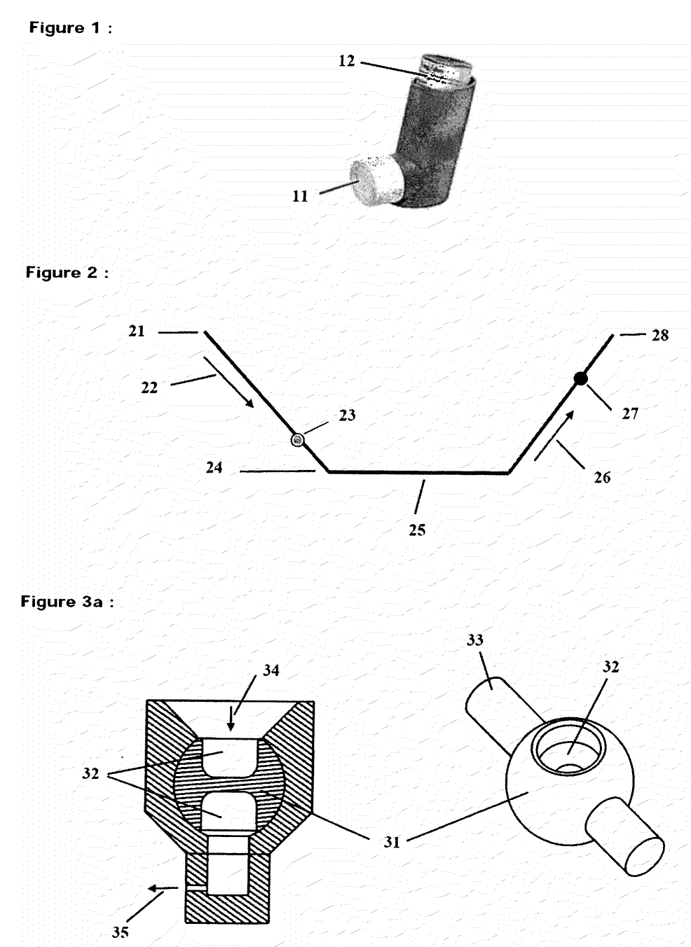

### DETAILED DESCRIPTION OF THE INVENTION

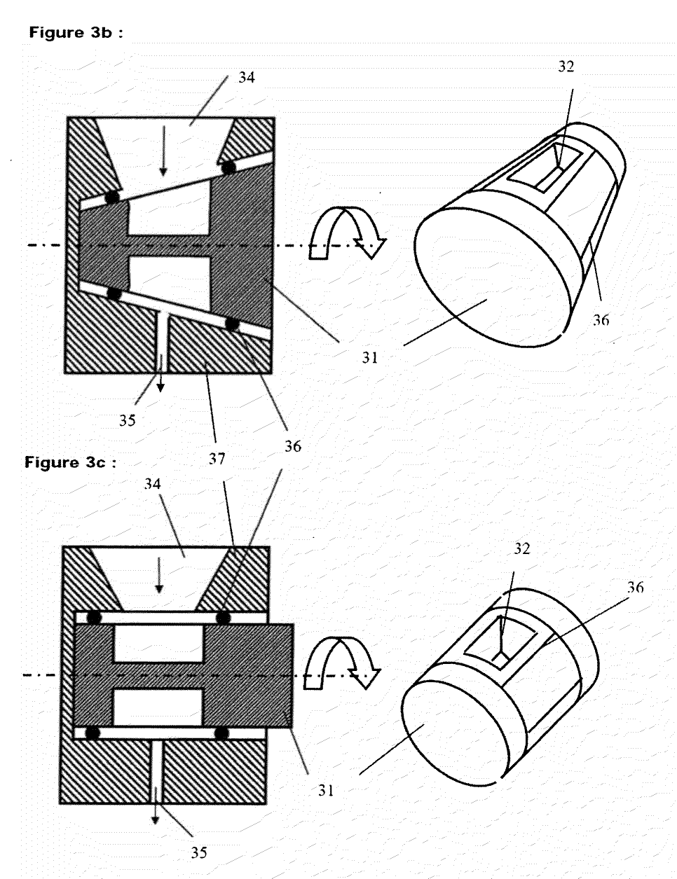

One possible embodiment of the forward metering valve disclosed in this invention is shown in FIG. 3a.

The valve rotor (31) shown is ball shaped. The valve rotor contains one or more metering cavities (32). During inhalation the valve rotor (33) is turned within the valve housing (37). In a first position the metering cavity is filled with the container medium through the container outlet (34). In a second position the metered volume in the cavity is enclosed by the wall of the valve housing (37). In a third position the metering cavity releases the dose to the patient through the outlet (35).

Other valve rotor shapes are possible, e.g., conically shaped FIG. 3b or cylindrically shaped FIG. 3c, as long as the shape is rotationally symmetric.

FIGS. 3b and 3c (36) indicates an example of a sealing structure needed for the valve member primarily to effectively seal the container outlet from the environment to avoid leakage during the full life time and secondly for effective metering and enclosing the dose during rotation until the valve reaches its dose release state. The sealing structure may be integral with the valve rotor (31) or integral with the valve housing (37).

FIGS. 3a, 3b and 3c all show embodiments, where the metering cavities (32) are placed within the valve rotors. It may, however, in some cases be advantageous to place the metering cavity in the valve housing structure. In this case the valve rotor acts merely as a fluid communication controller between the medium in the container, the metering cavity and the valve outlet to the nozzle.

The valve rotor may have a shaft as shown in FIG. 3a, (33) or may have a more alignment tolerant coupling mechanism such as a groove coupling or the like as shown in FIGS. 3b and 3c.

Rotational axes depend on the actual embodiment and may be horizontal or vertical or any angle in between.

Valve outlet (35) direction depends on the actual embodiment and may be horizontal or vertical or any angle in between.

Metering cavities may have any form, e.g., cylindrical, square formed, polygonal, and any combinations hereof.

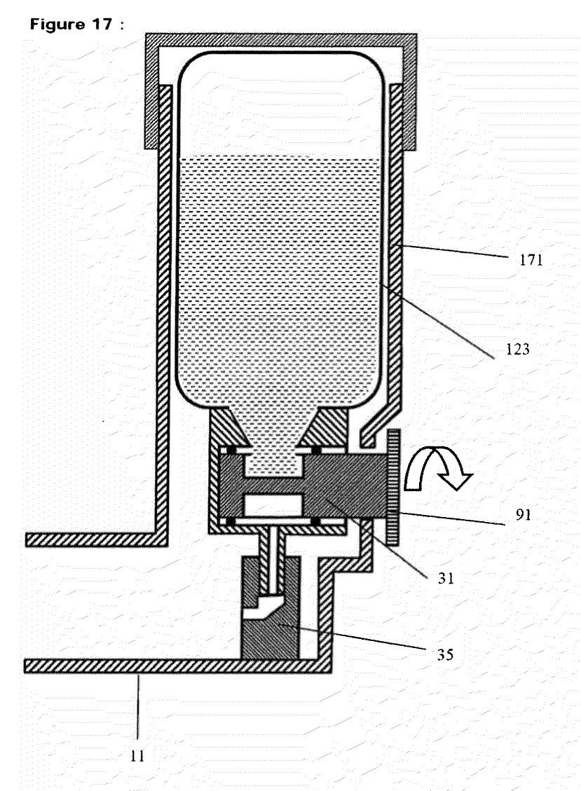

FIG. 17 shows an inhaler with an inhaler housing (171), a canister/container (123) and a rotational cylindrically shaped valve rotor (31), a nozzle (35), a mouthpiece (11) and a hand driven actuator wheel (91) in the simplest configuration.

The valve cycles during inhalation are shown in FIG. 4.

From an initial filling position (41) the valve rotor (31) is rotated clockwise to the metering position (42) where the metering chamber (32) is isolated from the inlet (34).

After passing the half-way position (43) where the metering chamber is fully closed to the surroundings, the dose release (44) happens when the metering chamber opens up towards the outlet. The last cycle is the stop position (45), which at the same time is the initial position for the next dose. The embodiment shown will rotate approximately 180° to release a dose (2 doses per 360° rotation). Other options are 1, 3, 4, 5, 6 and more doses per 360° rotation.

As the current dose is metered within seconds ahead of delivery, problems (C) and (D) are obviated. There will be no need for priming shots.

Due to the lack of a return spring the force to actuate the metering valve will be significantly lower than 30-50 N, and the effects of problem (A) will be significantly reduced.

As metering of the current dose is done during inhalation (forward metering), it is required to keep the inhaler upright during inhalation. This is far easier to understand for the patient than keeping the inhaler upright after inhalation, decreasing the effect of problem (B).

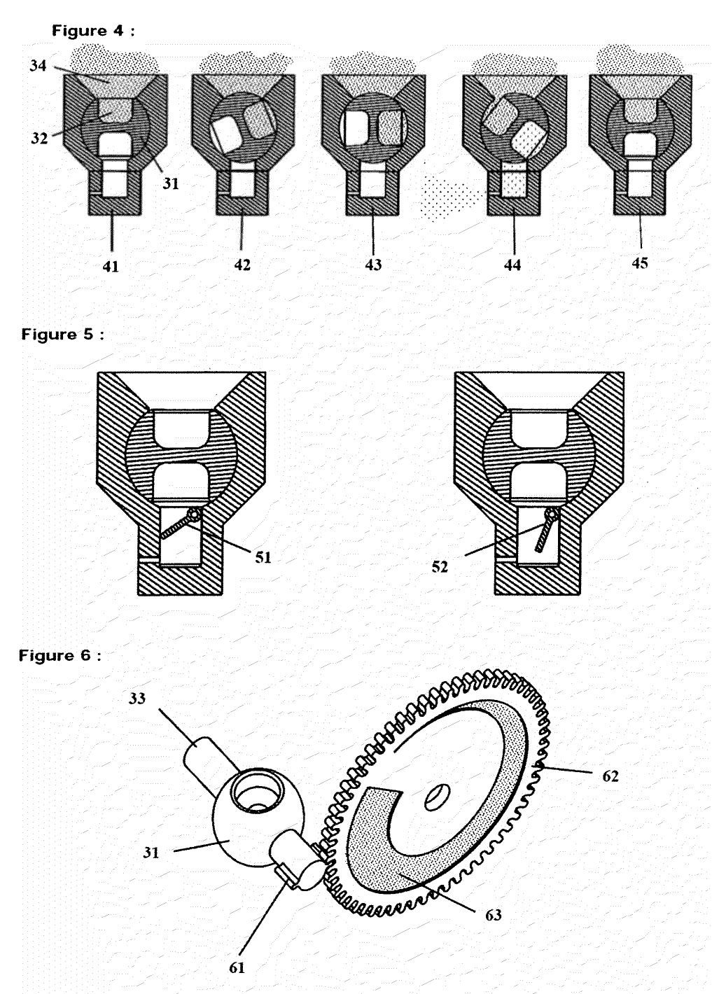

A potential problem with the proposed valve design is the possibility of feeding outside air and impurities into the pressurized drug bulk, when rotating an emptied metering chamber forward to the inlet position. This can be solved by adding a one-way valve to the outlet of the metering valve, preventing outside air to enter the emptied metering chamber. One possible embodiment of an additional one-way valve is shown in FIG. 5. Normally the one-way valve will be in its closed position (51), allowing no outside air to enter the metering chamber. During dose release, the one-way valve will open up (52), allowing the drug to escape from the metering chamber through the nozzle to the outside.

In the case of the metering valve being integral with the canister, the one-way valve may be placed in an attached nozzle member, still allowing for standard canister filling procedures.

Adding a dose indicator to the proposed valve design will be a simple task. Because the metering valve is only intended to move in one direction, the dose counter can be continuously engaged with the valve and synchronised with the valve movement, eliminating the position tolerance problem and the effects of tampering according to problem (E). One possible embodiment of a dose indicator is shown in FIG. 6.

The rotor gear wheel (61) is engaged with the indicator gear wheel (62), ensuring a fixed relation between the number of valve rotations and the position of the visual dose indicator (63). The visual dose indicator (63) can visualise the remaining drug level in the canister by a patterned or coloured field as shown, or it can be fitted with numbers or codes to indicate the approximate or precise number of doses left in the canister.

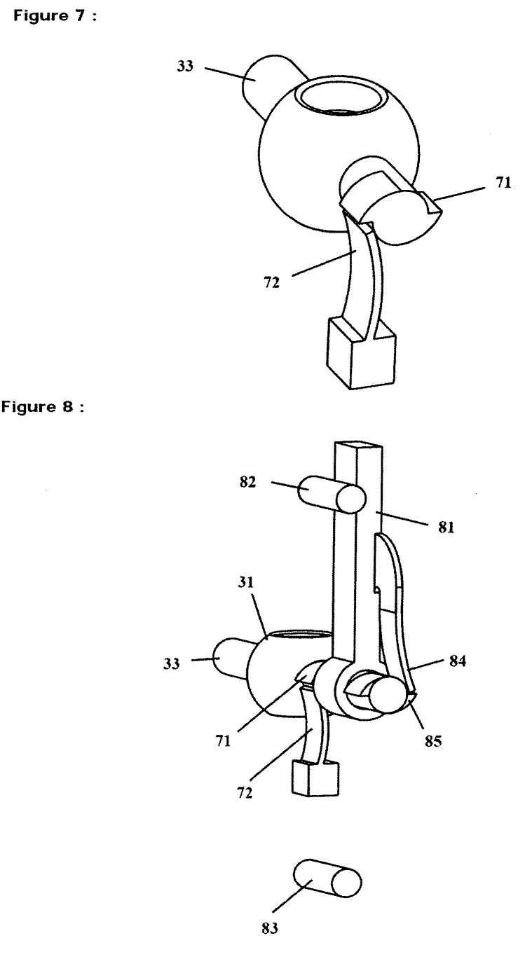

A potential risk of the proposed valve design in combination with the proposed dose indicator design is the risk of moving the valve backwards, releasing doses while turning the dose indicator backwards. This will lead to lack of synchronisation between the dose indicator status and the actual amount of drug left in the container, which is a serious malfunction of a drug dose indicator. One possible embodiment of a backwards lock is shown in FIG. 7.

When adding a backwards lock ratchet (71) to the rotor shaft (33) and a backwards lock spring (72) to the inhaler chassis it will become impossible to move the valve backwards, eliminating the risk of undercounting.

Another potential risk with the proposed valve design is the risk of releasing more doses than required per inhalation. To prevent this, a step lock can be applied. It will ensure that the valve will stop rotating after the required number of doses has been released during inhalation. The step lock can be realised in different embodiments. One possible option is shown in FIG. 8, releasing one dose per actuation.

The valve actuator (81) is mounted free-rotating on the rotor shaft (33). To actuate the valve and release one dose, the valve actuator must be moved clockwise from its upright position resting against the actuator reverse stop (82) to it's downwards position stopped by the actuator forward stop (83). During this, the step lock spring (84) will engage the step lock ratchet (85), rotating the rotor shaft (33) and the valve rotor (31) forward. To prepare the valve for the next dose, the valve actuator (81) must be returned to its upright position, resting against the actuator reverse stop (82). During this, the backwards lock spring (72) will engage the backwards lock ratchet (71), ensuring that the valve rotor (31) will not rotate backwards.

Delivering a single dose with the rotational valve requires a rotational input to the valve shaft to actuate the valve during inhalation. Basically, the valve rotation can be actuated in two different ways:

1. Manual actuation

2. Breath actuation

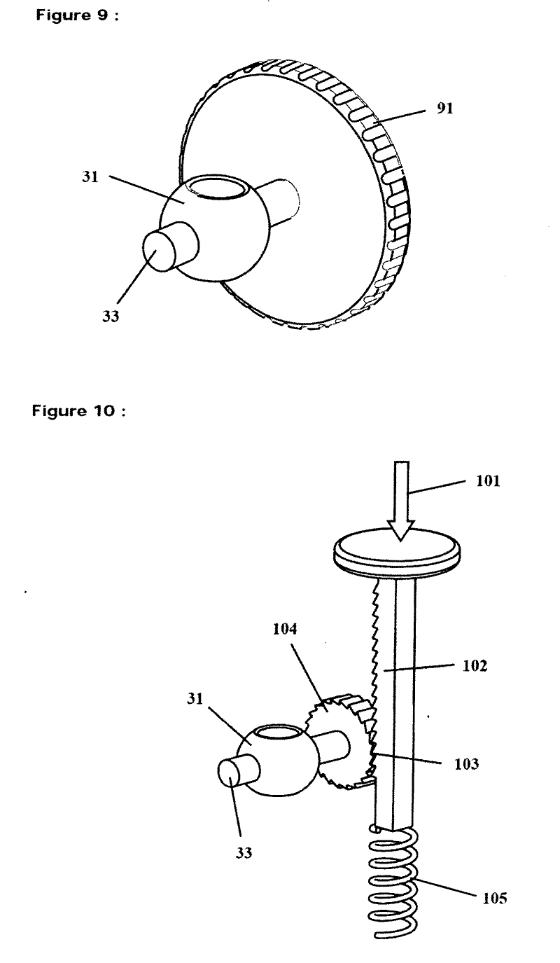

Manual actuation can be obtained by requiring the user to manually actuate the valve rotation. One possible embodiment is shown in FIG. 9, where a finger wheel (91) is mounted directly onto the rotor shaft (33) to directly rotate the valve rotor (31) and hereby releasing a dose of drug.

Another possible embodiment is shown in FIG. 10, where a mechanism requires the user to perform a linear input movement (101) to rotate the valve rotor (31). During user input a rack (102) travels down. The pinion (103) is engaged with a rotor gear wheel (104) mounted on the rotor shaft (33), causing the valve rotor (31) to rotate clockwise and hereby releasing a dose of drug. After inhalation a return spring (105) can cause the rack (102) to return to the initial position without causing the valve rotor (31) to rotate counter-clockwise, by performing as a ratchet.

Breath actuation can be obtained by using stored energy to actuate the valve rotation.

The stored energy is triggered by the user's inhalation through the inhaler. The energy can be stored in several ways.

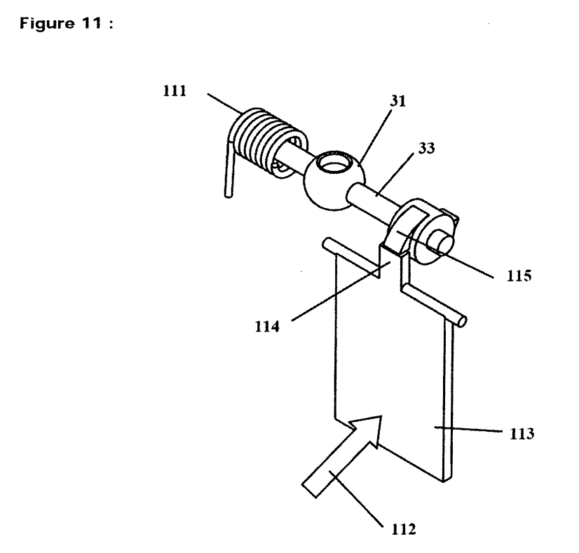

In FIG. 11 one possible embodiment is shown, where energy stored in a loaded spring (111) is applied to the rotor shaft (33), directly rotating the valve rotor (31) counter-clockwise and hereby releasing a dose of drug. To trigger the loaded spring (111) by the user's inhalation flow (112) a hinged flap (113) is mounted in the airflow path of the inhaler, causing the flap lock (114) to release the rotor lock (115). Hereby the loaded spring (111) is allowed to rotate the valve rotor (31) and hereby release a dose of drug. Instead of the activation by the hinged flap (113) a simple push button may be used to release the rotor lock

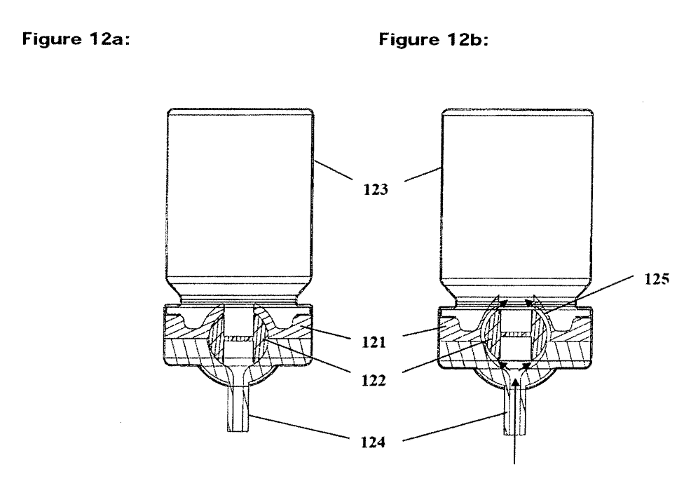

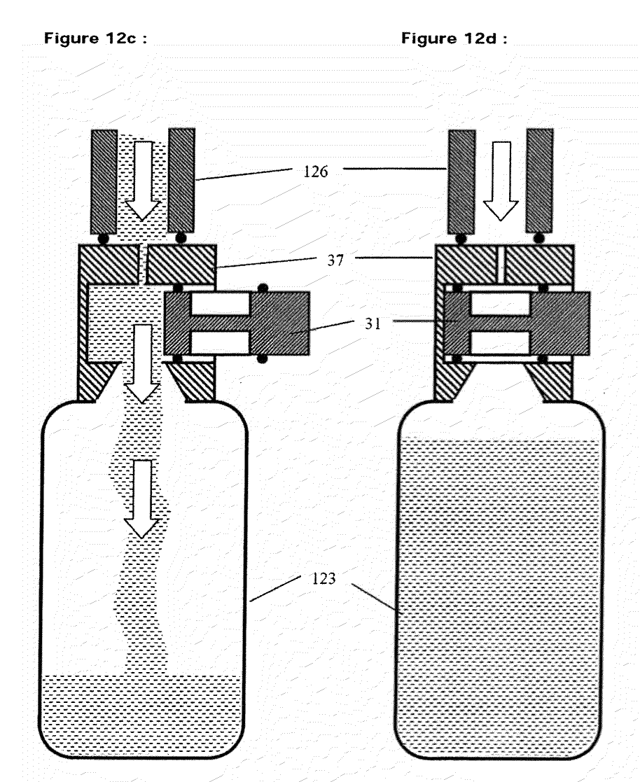

FIG. 12 shows one embodiment of a ball shaped forward metering valve (122) being integral with a canister (123). In this embodiment with two metering cavities one inhalation procedure results in a 180° rotation of the valve. In the case of the forward metering valve being integral with the canister, the valve must allow for reverse flow through the valve, whenever a pressurized liquid is applied to the valve outlet (124). One embodiment is shown on FIG. 12a, where an elastic sealing member (121) effectively seals the pressurized liquids in the canister (123) from leaking through the valve. In the canister filling situation on FIG. 12b, the pressure at the valve outlet (124) is higher than the pressure inside the canister. This pressure difference will force the sealing member to open (125) in the same way as an inflation valve in a bicycle inner tube and therefore allow the pressurized liquid to flow into the canister until an equilibrium pressure is present across the valve. Problem (F) is therefore solved.

FIGS. 12c and 12d show another solution to the filling requirement F. In this embodiment a cylindrical valve rotor (31) is used and the valve is integral with the container. During assembly of the valve, the valve rotor (31) is not fully inserted into the valve housing (37), thereby enabling the filling of the pressurized medium through the port (126). When the container is filled the valve rotor (31) may be fully inserted, thereby enclosing the medium in the container (12) and the filling port (121) can be removed.

A further aspect of the invention is that the forward metering valve disclosed here is extremely suitable for multi-dose operation, because the mechanical movement of the valve is rotational and unidirectional. The metering cavities can be filled and emptied during rotation at reasonable turning speeds, thus allowing several metered doses to be released during an inhalation sequence.

This aspect unfolds several applications that could solve some problems related to administration of drugs:

Pulmonary administration of insulin is a promising new drug delivery therapy. Unlike most asthma inhalers that deliver the same dose every time, insulin inhalers must be able to preset and deliver different dose sizes dependent of time of day, meals intake, and exercise levels.

Pulmonary administration of pain killers for patients having chronic pain also requires adjustment of doses to the actual pain level.

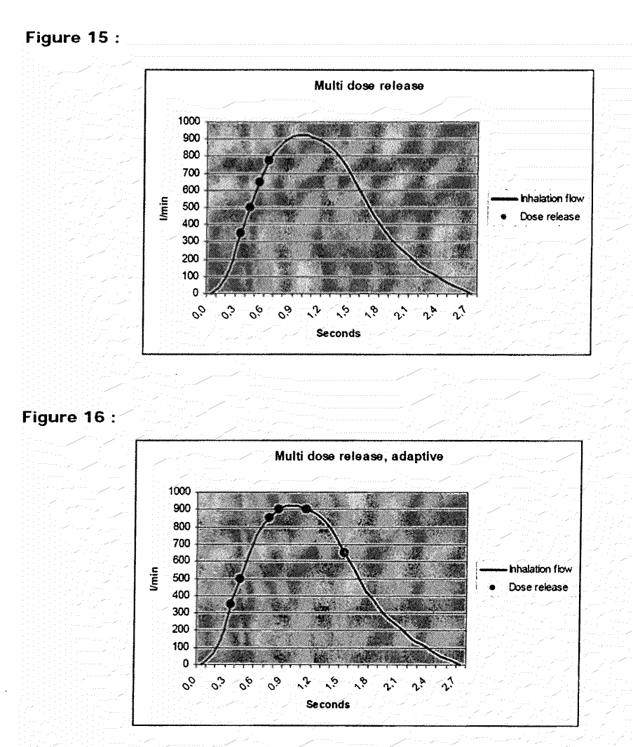

Inhaler research has indicated that it is advantageous for optimal drug medium deposition to release smaller dose portions during the inhalation sequence shown in FIG. 15 instead of one big puff in the beginning of the inhalation sequence shown in FIG. 14. This can easily be achieved by the forward metering valve by choosing the right relation between metering cavity volume, valve rotation speed and number of released doses. The arrangement in FIG. 10 may easily be supplemented by a preset blocking mechanism that will only allow the rack (101) to move to a certain preset position, where the preset number of doses has been released.

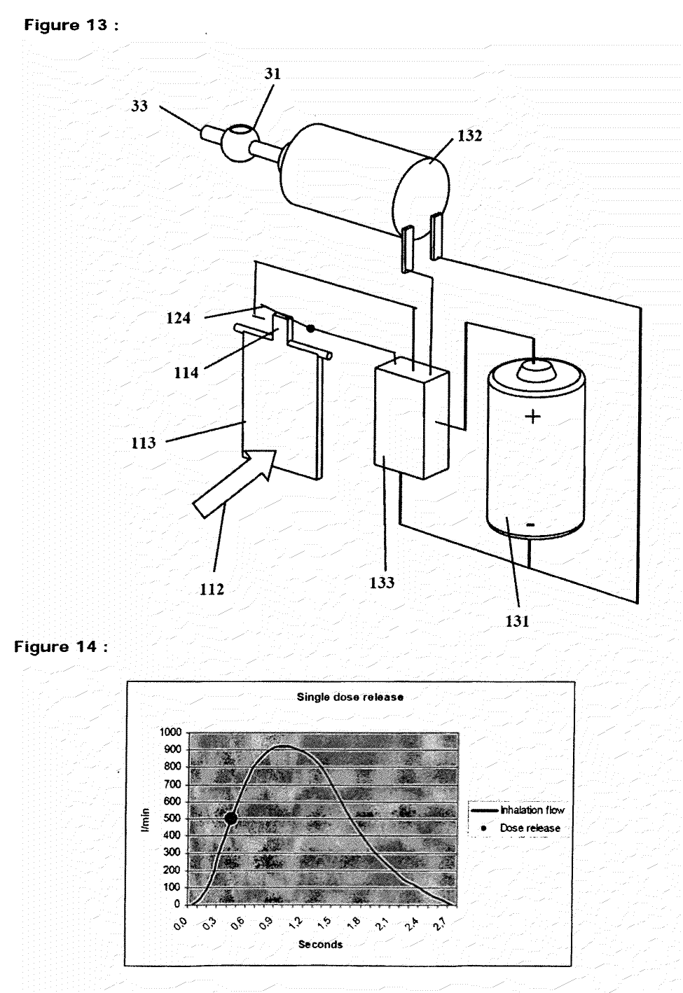

A further aspect of the invention is that the unidirectional rotation of the forward metering valve is easily connected to and driven by a simple motor as shown in FIG. 13. The battery (131) powered motor (132) may be controlled by timing alone or more advantageously by measuring the actual inhaler flow in the flow channel and adapt the dose release pattern to the actual inhalation flow profile FIG. 16. The flow sensor could i.e. be of the differential pressure type, hot wire anemometry type or even a mechanical displacement type sensor (113). Besides valve release control the controller (133) might also solve safety issues like reliable dose counting and overdose protection by disabling dose releases in a certain period of time after a successful inhalation sequence has been performed.

Summing up, the invention relates to a metered dose inhaler for administration a liquid phase medium. It uses a rotating metering element that transports the metered dose from the pressurized canister to the mouthpiece. The metering is improved in that there is an efficient protection against penetration of outside air, and in that the counting of the doses is improved by preventing backwards rotation of the rotating metering element.

The invention has been described in some detail above, but this is not limiting per se, as the skilled person will be able to devise additional mechanical solutions that perform in an equivalent manner, thereby obtaining similar advantageous results.

The foregoing description of the specific embodiments will so fully reveal the general nature of the present invention that others skilled in the art can, by applying current knowledge, readily modify o r adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of forms without departing from the invention.

Thus, the expressions “means to . . . ” and “means for . . . ”, or any method step language, as may be found in the specification above and/or in the claims below, followed by a functional statement, are intended to define and cover whatever structural, physical, chemical, or electrical element or structure, or whatever method step, which may now or in the future exist which carries out the recited functions, whether or not precisely equivalent to the embodiment or embodiments disclosed in the specification above, i.e., other means or steps for carrying out the same function can be used; and it is intended that such expressions be given their broadest interpretation.

What is claimed is:

1. A pressurized metered dose inhaler having a housing for holding a container with a pressurized medium, a spray nozzle for aerosolization of the medium to be inhaled, and a metering valve with a rotatable valve member for controlling fluid communication between the container and the nozzle, characterized in the metering valve having at least one metering cavity, the valve having a seal between the valve member and valve housing for sealingly connecting the valve with an output of the pressurized container, the valve member being rotationally positioned in a valve housing, for, the valve member in a first position, receiving a dose of the medium into one of the at least one metering cavities, and in a second position enclosing the metering cavity, and a third position, discharging a dose of the medium from the metering cavity.

2. An inhaler according to claim 1, characterized in that the metering cavity is incorporated in the rotatable valve member.

3. An inhaler according to claim 1, characterized in that the metering cavity is incorporated in the valve housing.

4. A metering valve according to claim 1, characterized in that the valve rotor is built into the structure of a pressurized canister.

5. An inhaler according to claim 1, for the valve member in said first position, the pressure from a pressurized medium applied to the valve outlet will deflect the sealing and valve member, thereby facilitating filling of said container.

6. A procedure for filing of a pressurized metered dose inhaler having a housing for holding a container with a pressurized medium, a spray nozzle for aerosolization of the medium to be inhaled, and a metering valve with a rotatable valve member for controlling fluid communication between the container and the nozzle, characterized in the metering valve having at least one metering cavity, the valve having a seal between the valve member and valve housing for sealingly connecting the valve with an output of the pressurized container, the valve member being rotationally positioned in a valve housing, for, the valve member in a first position, receiving a dose of the medium into one of the at least one metering cavities, and in a second position enclosing the metering cavity, and a third position, discharging a dose of the medium from the metering cavity, comprising the steps of: partially inserting the valve member in a first state into the valve housing, filling of said container, followed by producing of a second state, where the valve member is fully inserted into the valve housing and seals the filled container from the environment, whenever the valve member is kept in a stationary position.

7. An inhaler according to claim 1, characterized in that the said rotational valve is rotationally symmetrically shaped.

8. An inhaler according to claim 1, characterized in that a further one-way valve is arranged between the valve rotor and the outlet, where the one-way valve prevents a medium, such as atmospheric air or vapor, from flowing into the metering chamber through the outlet.

9. An inhaler according to claim 1, characterized in that a valve rotor shaft is connected to a visual dose indicator, said visual dose indicator displaying the approximate number of doses left in the said container.

10. An inhaler according to claim 1, characterized in that a valve rotor is connected to locking means, said locking means comprising a locking spring, said locking spring interacting with a backwards locking ratchet in order to ensure a one-way rotation of the valve rotor.

11. An inhaler according to claim 1, characterized in that a valve rotor is connected to mechanical transmission means, said mechanical transmission means connected to activation means for generating a rotational movement of the valve rotor, when said activation means are activated.

12. An inhaler according to claim 1, characterized in that the inhaler comprises an activation means, such as a button or the like, said activation means connected to actuating means.

13. An inhaler according to claim 12, characterized in that the said activation means comprises a breath actuated member, said member connected to trigger means for controlling the said actuating means.

14. An inhaler according to claim 1, characterized in that the inhaler comprises means for controlling the number of doses or sub-doses for each inhalation, which controlling means ensures that the valve rotor stops rotating after a defined number of doses or sub-doses.

15. A procedure for operating an inhaler having a housing for holding a container with a pressurized medium, a spray nozzle for aerosolization of the medium to be inhaled, and a metering valve with a rotatable valve member for controlling fluid communication between the container and the nozzle, characterized in the metering valve having at least one metering cavity, the valve having a seal between the valve member and valve housing for sealingly connecting the valve with an output of the pressurized container, the valve member being rotationally positioned in a valve housing, for, the valve member in a first position, receiving a dose of the medium into one of the at least one metering cavities, and in a second position enclosing the metering cavity, and a third position, discharging a dose of the medium from the metering cavity comprising the following operational steps:
an initial step, where a metering chamber is connected to a volume containing a medium under pressure,
a second step, where the valve rotor is rotated to enclose the metering chamber, and
a third step, where the valve rotor is further rotated so the medium in the metering chamber is connected to the outlet nozzle.

16. A procedure for operating an inhaler according to claim 15 further comprising the following steps:
an activation step initiated by a user/patient,
an inhalation step where the user/patient inhales and where the valve rotor is rotated in given number of turns, in such a way that at least one metering chamber is emptied a given number of times in order to deliver a predetermined dose of medicine to the user/patient.
